(19) United States
(12) Patent Application Publication
(10) Pub. No.: US 2024/0428886 A1
(43) Pub. Date: Dec. 26, 2024

(54) COMPUTERIZED SYSTEMS AND METHODS FOR ENSEMBLE MODEL-BASED DRUG DISCOVERY

(71) Applicant: Lantern Pharma Inc., Dallas, TX (US)

(72) Inventors: Joseph McDermott, Dallas, TX (US); Panna Sharma, Atlanta, GA (US); Umesh Kathad, Dallas, TX (US)

(73) Assignee: Lantern Pharma Inc., Dallas, TX (US)

(21) Appl. No.: 18/829,920

(22) Filed: Sep. 10, 2024

Related U.S. Application Data
(63) Continuation of application No. PCT/US2023/064050, filed on Mar. 9, 2023.
(60) Provisional application No. 63/269,148, filed on Mar. 10, 2022.

(51) Int. Cl. G16B 20/50; G16B 40/00; G16B 50/30; G16H 20/10

(52) U.S. Cl. CPC G16B 20/50; G16B 40/00; G16B 50/30; G16H 20/10

(57) ABSTRACT

Disclosed are systems and methods that provide a novel framework for decision intelligence (DI)-based drug determinations. The disclosed framework can leverage a dynamically and recursively trained artificial intelligence/machine learning (AI/ML) ensemble configuration to analyze genomic data and functions that arrive from the same. Ensemble determinations and applications can increase the accuracy of the training, validation, and external testing sets associated with drug discovery and personalization. The ensemble-based computerized framework can be configured for analysis of samples using an ensemble algorithm trained with a binary mutation data and a hierarchical clustering data, which can enable determinations of drug efficacy and patent stratification.

### CROSS-REFERENCE TO RELATED APPLICATIONS

This application is a continuation of International Patent Application No. PCT/US23/64050, filed on Mar. 9, 2023, which claims the benefit of priority to U.S. Provisional Patent Application No. 63/269,148, filed on Mar. 10, 2022, each of which is incorporated by reference in its entirety herein.

### FIELD OF THE DISCLOSURE

The present disclosure is generally related to a drug efficacy prediction and patient stratification, and more particularly, to a computerized framework for leveraging applications of a decision-intelligence (DI) based model for drug discovery and applicability monitoring.

### BACKGROUND

Over the past few years, there has been a drastic increase in data digitization in the pharmaceutical sector. However, such digitalization involves, among other drawbacks, the challenge of acquiring, scrutinizing and applying that knowledge to solve complex clinical problems.

### SUMMARY OF THE DISCLOSURE

According to some embodiments, disclosed are systems and methods for a novel computerized framework for DI-based drug determinations based on such digitization. As discussed herein, the computerized framework can leverage a dynamically and recursively trained artificial intelligence/machine learning (AI/ML) ensemble configuration to analyze genomic data and functions that arrive from the same. According to some embodiments, ensemble determinations and applications can increase the accuracy of the training, validation, and external testing sets associated with drug discovery and personalization. Moreover, in some embodiments, the ensemble-based computerized framework can be configured for analysis of samples using an ensemble algorithm trained with a binary mutation data and a hierarchical clustering data, which can enable determinations of drug efficacy and patent stratification.

Accordingly, as evident from the instant disclosure, usage of the ensemble-based algorithmic approach provides an improved accuracy as compared to a single algorithm's usage (e.g., on the same data). For example, in predicting a patient's drug response, an ensemble approach can evidence a 7.7% increase in accuracy of drug selection and dosage identification for a particular patient. Thus, more robust, accurate analyses and determinations are driven via the ensemble configurations, as discussed herein.

As discussed herein, an ensemble Models refers to a multi-layer combination of AI/ML (and/or deep learning) algorithms that combines predictions of other algorithms as features used to train a final algorithm and produce the final ensemble. The ensemble of predictors can be individually trained and have their hyperparameters tuned for optimal performance. Conceptually, the ensemble allows the higher-layer algorithm to learn from the performance of the lower layer. In addition, the nature of an ensemble provides a solution to several common problems in machine learning. Thus, when tuning a single algorithm, there may be contexts where one set of hyperparameters is optimal, yet there may be frequent exceptions which lead to inaccurate results. However, as discussed herein, in an ensemble, algorithms (and/or sets of algorithms) can be repeated many times, with different tuning, thereby expanding the functionality and applicability of drug selection and application to patient's conditions.

According to some embodiments, a method is disclosed for a DI-based computerized framework for deterministically monitoring and tracking identified drug applicability to a patient's condition. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for a DI-based computerized framework for deterministically monitoring and tracking identified drug applicability to a patient's condition.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary workflow according to some embodiments of the present disclosure;

FIG. 4 depicts a non-limiting ensemble model configuration according to some embodiments of the present disclosure;

FIG. 5 depicts an exemplary implementation of an architecture according to some embodiments of the present disclosure;

FIG. 6 depicts an exemplary implementation of an architecture according to some embodiments of the present disclosure; and

FIG. 7 is a block diagram illustrating a computing device showing an example of a client or server device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3^{rd}, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments and principles will be discussed in more detail with reference to the figures. According to some embodiments, as discussed herein, the disclosed systems and methods provide computerized mechanisms for the analysis of genomic data and functions that arrive from the same. As discussed herein, the ensemble-based computerized framework can be configured for analysis of samples using an ensemble algorithm trained with a binary mutation data and a hierarchical clustering data, which can enable determinations of drug efficacy and patent stratification. In some embodiments, as discussed below, such drug efficacy and patient stratification can be based on determined scores corresponding to drug responsiveness to a patient's condition(s).

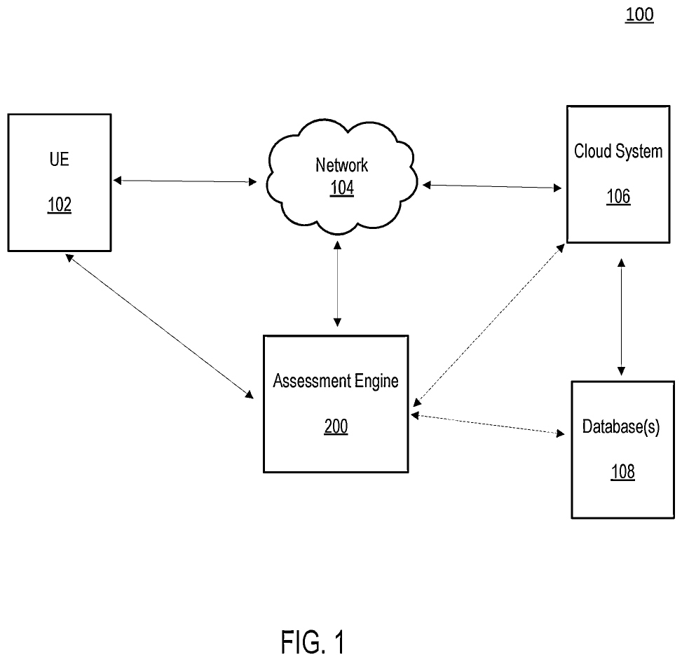

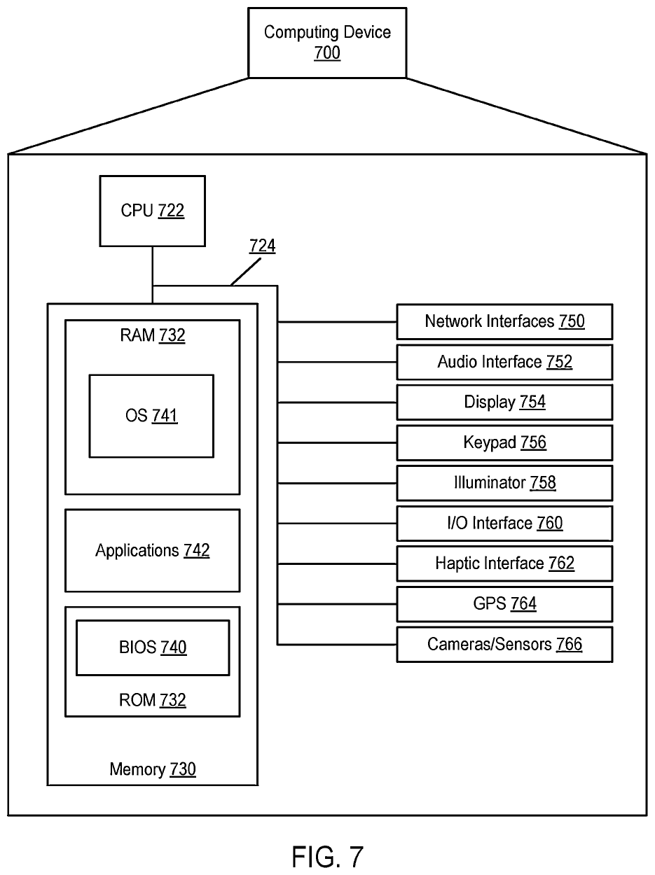

With reference to FIG. 1, system 100 is depicted, which according to some embodiments, can include user equipment (UE) 102 (e.g., a user device, as mentioned above and discussed below in relation to FIG. 7), network 104, cloud system 106, database(s) 108 and assessment engine 200. It should be understood that while system 100 is depicted as including such components, it should not be construed as limiting, as one of ordinary skill in the art would readily understand that varying numbers of UEs, cloud systems, databases and/or networks can be utilized without departing from the scope of the instant disclosure; however, for purposes of explanation, system 100 is discussed in relation to the example depiction in FIG. 1.

According to some embodiments, UE 102 can be any type of device, such as, but not limited to, a mobile phone, tablet, laptop, sensor, wearable device, wearable camera, wearable clothing, a patch, Internet of Things (IoT) device, autonomous machine, and any other type of modern device. In some embodiments, UE 102 can be a device associated with an individual (or set of individuals) for which drug responsiveness monitoring are being provided.

In some embodiments, UE 102 can be associated with a peripheral device (not shown), which can be connected to UE 102, and can be any type of peripheral device, such as, but not limited to, a wearable device (e.g., smart watch), printer, speaker, sensor, and the like. In some embodiments, peripheral device can be any type of device that is connectable to UE 102 via any type of known or to be known pairing mechanism, including, but not limited to, WiFi, Bluetooth™, Bluetooth Low Energy (BLE), NFC, and the like.

In some embodiments, network 104 can be any type of network, such as, but not limited to, a wireless network, cellular network, the Internet, and the like (as discussed above). Network 104 facilitates connectivity of the components of system 100, as illustrated in FIG. 1.

According to some embodiments, cloud system 106 may be any type of cloud operating platform and/or network based system upon which applications, operations, and/or other forms of network resources may be located. For example, system 106 may be a service and/or health provider, and/or network provider from where services and/or applications may be accessed, sourced or executed from. For example, system 106 can represent the cloud-based architecture associated with a healthcare provider, which has associated network resources hosted on the internet or private network (e.g., network 104), which enables (via engine 200) the patient monitoring and management discussed herein.

In some embodiments, cloud system 106 may be a private cloud, where access is restricted by isolating the network such as preventing external access, or by using encryption to limit access to only authorized users. Alternatively, cloud system 106 may be a public cloud where access is widely available via the internet. A public cloud may not be secured or may include limited healthcare features.

In some embodiments, cloud system 106 may include a server(s) and/or a database of information which is accessible over network 104. In some embodiments, a database 108 of cloud system 106 may store a dataset of data and metadata associated with local and/or network information related to a user(s) of UE 102 and the UE 102, and the services and applications provided by cloud system 106 and/or assessment engine 200.

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

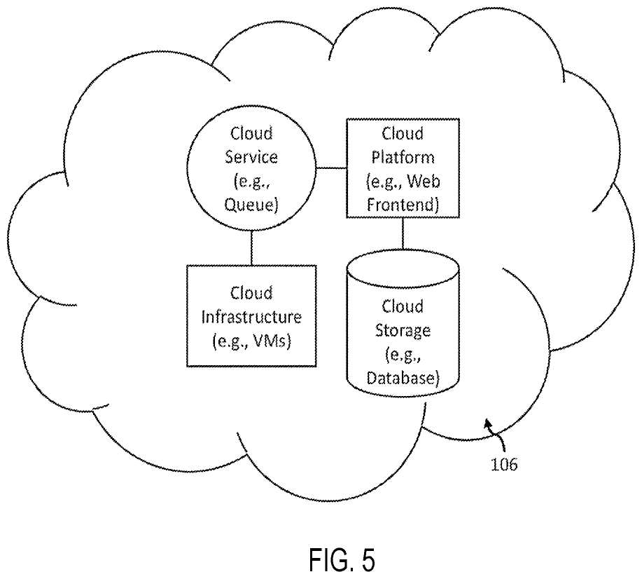

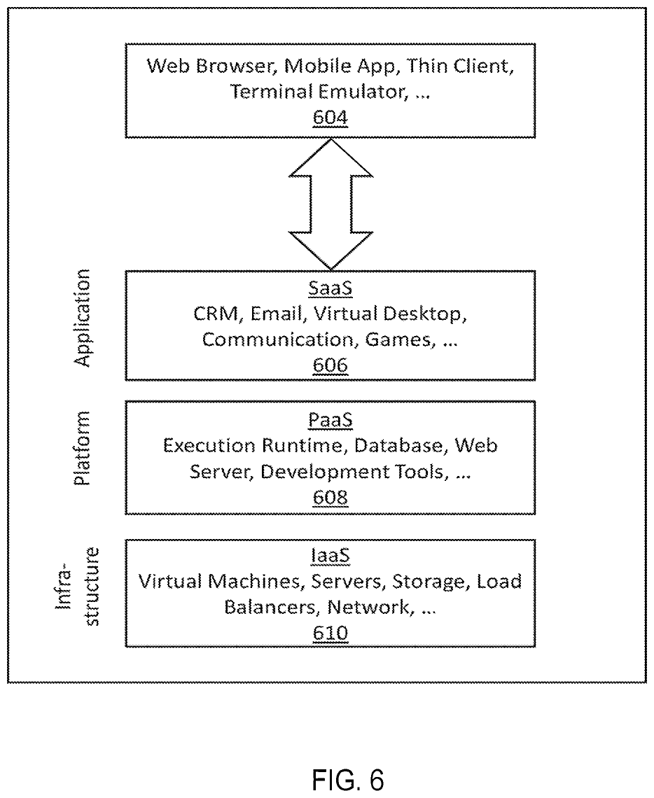

Turning to FIGS. 5-6, in some embodiments, the exemplary computer-based systems/platforms, the exemplary computer-based devices, and/or the exemplary computer-based components of the present disclosure may be specifically configured to operate in a cloud computing/architecture 106 such as, but not limiting to: infrastructure a service (IaaS) 610, platform as a service (PaaS) 608, and/or software as a service (SaaS) 606 using a web browser, mobile app, thin client, terminal emulator or other endpoint 604. FIGS. 5-6 illustrate schematics of non-limiting implementations of the cloud computing/architecture(s) in which the exemplary computer-based systems for administrative customizations and control of network-hosted APIs of the present disclosure may be specifically configured to operate.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, standard query language (SQL). According to some embodiments, database 108 may correspond to any type of known or to be known type of storage, such as, but not limited to a, look-up table (LUT), distributed ledger of a distributed network, and the like.

Assessment engine 200, as discussed above and further below in more detail, can include components for the disclosed functionality. According to some embodiments, assessment engine 200 may be a special purpose machine or processor, and can be hosted by a device on network 104, within cloud system 106 and/or on UE 102. In some embodiments, engine 200 may be hosted by a server and/or set of servers associated with cloud system 106.

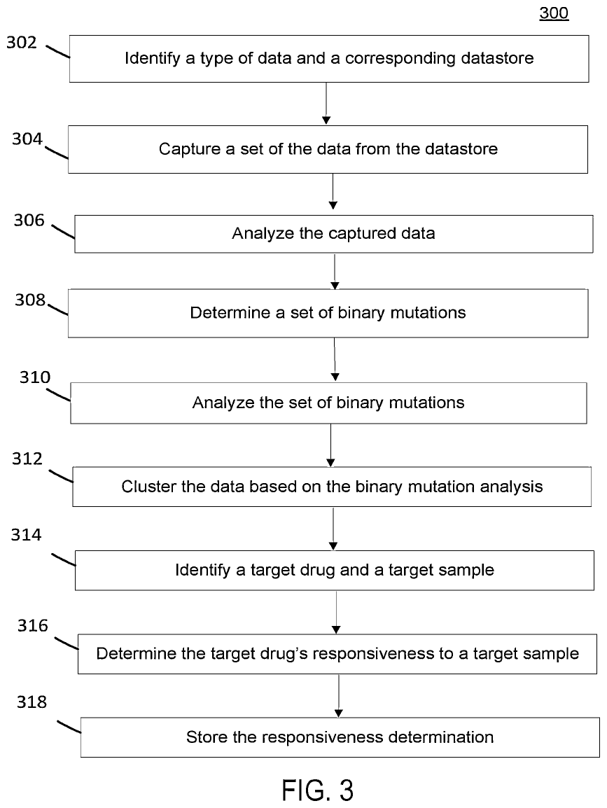

According to some embodiments, as discussed in more detail below, assessment engine 200 may be configured to implement and/or control a plurality of services and/or microservices, where each of the plurality of services/microservices are configured to execute a plurality of workflows associated with performing the disclosed drug discovery and patient monitoring and management. Non-limiting embodiments of such workflows are provided below in relation to at least FIG. 3.

According to some embodiments, as discussed above, assessment engine 200 may function as an application provided by cloud system 106. In some embodiments, engine 200 may function as an application installed on a server(s), network location and/or other type of network resource associated with system 106. In some embodiments, assessment engine 200 may function as an application operating via a conventional edge device (not shown) at location associated with system 100. In some embodiments, engine 200 may function as application installed and/or executing on UE 102. In some embodiments, such application may be a web-based application accessed by UE 102 over network 104 from cloud system 106. In some embodiments, engine 200 may be configured and/or installed as an augmenting script, program or application (e.g., a plug-in or extension) to another application or program provided by cloud system 106 and/or executing on UE 102.

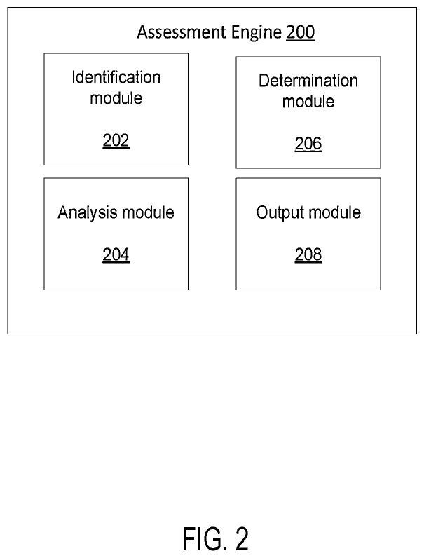

As illustrated in FIG. 2, according to some embodiments, assessment engine 200 includes identification module 202, analysis module 204, determination module 206 and output module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

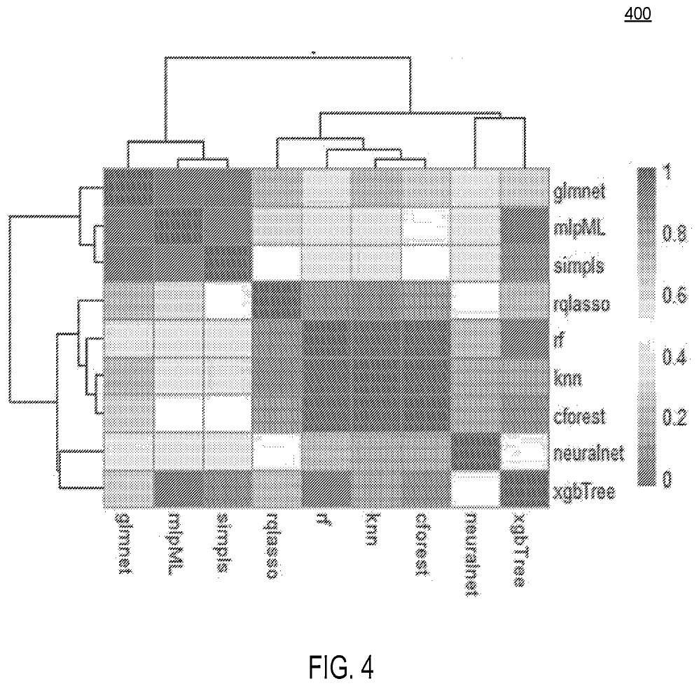

Turning to FIG. 3, Process 300 provides non-limiting example embodiments for the disclosed framework. According to some embodiments, Process 300 provides computerized mechanisms for the analysis of genomic data and functions that arrive from the same. As discussed herein, the ensemble-based computerized framework can be configured for analysis of samples using an ensemble algorithm (see, FIG. 4, discussed infra) trained with a binary mutation data and a hierarchical clustering data, which can enable determinations of drug efficacy and patent stratification.

According to some embodiments, Steps 302-304 and 314 of Process 300 can be performed by identification module 202 of assessment engine 200; Steps 306 and 310 can be performed by analysis module 204; Steps 308, 312 and 316 can be performed by determination module 206; and Step 318 can be performed by output module 208.

According to some embodiments, Process 300 begins with Step 302 where engine 200 can identify a type of data and a corresponding datastore (e.g., a database(s) that houses such specific type of data). For example, Step 302 can involve engine 200 receiving a request from a user for the identification of genomic data from a public and/or private database. In some embodiments, the data sets stored in such databases can include, but are not limited to, genomic data, mutation data, clinical outcome data (e.g., data representing the outcome of a treatment, for example), treatment data, drug response data (e.g., half-maximal inhibitory concentration (IC50) data), molecular data, patient identifiers, demographics, biometrics, and the like, or some combination thereof. Accordingly, such databases can store multiple distinct data sets derived from genomics data of multiple distinct diseased cells, respectively, where each data set can include a plurality of pathway element data.

In Step 304, engine 200 can query the identify database in order to capture, retrieve, extract or otherwise identify the specifically requested type (and/or quantity) of data. Thus, in Step 304, the requested data can be retrieved or captured from the database.

In Step 306, engine 200 can analyze the captured data, where in Step 308, based on the analysis of Step 306, a set of binary mutations can be determined. That is, in some embodiments, engine 200 can receive the queried distinct data sets from the database, and identify a determinant pathway element (or feature, used interchangeably) in the distinct data sets that is associated with a status (e.g., sensitive or resistant) of a treatment parameter (e.g., treatment with a drug) of the diseased cells.

According to some embodiments, as discussed herein, pathway elements/features can be derived from binary values representing mutation status as 0 or 1 into a continuous value based on the fraction of mutations affecting the same biological pathway. By way of example, if 4 mutations functionally contribute to the same biological function, and 2 of these are mutated in one tumor sample, a pathway feature can be generated with a score such as 2/4 or 0.5 for that sample's pathway feature.

Consequently, instead of many sparse data features, typically of mostly 0s and few 1s, engine 200 can create information rich features on a continuous gradient of values. Notably, it may not be possible for a mutation occurring at an average frequency to be used in AI/ML environments because in a realistic sample size (for example, a sample size of 100), an occurrence of 5 mutations for a gene is too few for any currently known AI/ML algorithms to train with cross validation and split into a test set (note, this may change upon the discovery of more independent and less training-dependent AI/ML). As discussed herein, pathway feature engineering expands the number of features with enough variation to be used by 10-50 fold. The result is more features that are usable, and that are of higher information content. Thus, reliance on feature engineering (as provided herein) in addition to mutations (rather than mutations alone) in AI/ML environments enables the performance of predictions of drug responses, which without the pathway information, is not a viable process.

In some embodiments, pathway-based features can be created from pathways that are organized to contain consistent effectors, when derived from a database, rather than clustering. For example, it may be common for some pathways commonly used in biological analyses to contain activators and suppressors of a single activity in a list of pathway genes, which is not suitable for AI/ML feature calculation—for example, derived pathways would be incoherent. For example, a score of 0.5 from such a pathway could mean mutations in 2 suppressors, or 2 activators, or 1 of each. Their inconsistency would yield an ambiguous feature that does not necessarily associate with a single functional impact. Thus, pathways with consistent feature lists, like, for example, the human REACTOME database (reactome.org), or from experimental protein-protein interactions, inter alia, can be used as the source of lists for pathway feature calculation.

According to some embodiments, scores can be calculated from original binary mutations to represent the degree of binary mutations in a pathway, which can be normalized by the length of the pathway. This divides the total sum of binary mutations by the length of the pathway available to give the feature score in one sample. The length available is the length of the number of genes in a pathway after removing the genes that were not available in the original data.

By way of a non-limiting example, in data from assays where only 350 genes are measured, many pathways have genes that are not tested, and so these are removed from the calculation to undue indirect influence of the measurement technology on feature weight. Accordingly, the disclosed methodologies can provide mechanisms for standardizing across different technologies (e.g., any technology or art where feature extraction and reduction can be utilized to focus in on viable elements for AI/ML processing, for example). In some embodiments, for example, pathway features are robust whether genomic data are comprised of 300, 500, or over 20000 genes.

Thus, the query in Step 304, for example, can have conditions corresponding to the elements of/for pathway engineering (e.g., pathway elements/features), a status and/or a treatment parameter, for example.

In some embodiments, in addition to the feature engineering discussed above, Steps 306-308 can additionally (or alternatively, in some embodiments) involve the analysis of distinct data sets associated with diseased cells, and the determination of pathway elements in the data set that are modulated to produce a modified data set. Accordingly, in some embodiments, engine 200 can leverage the modified data set to identify a change in status of the treatment parameter for the diseased cell. In some embodiments, where desirable and/or needed, engine 200 may pre-process the datasets (e.g., feature selection, data transformation, metadata transformation, and/or splitting into training and validation datasets) prior to the analysis of Step 306.

In some embodiments, as mentioned above, such analysis and determination (e.g., Steps 306-308) can be performed by engine 200 utilizing any type of known or to be known AI/ML algorithm or technique including, but not limited to, computer vision, classifier, feature vector analysis, decision trees, boosting, support-vector machines (SVMs), neural networks (e.g., convolutional neural network (CNN), recurrent neural network (RNN), and the like), nearest neighbor algorithms, Naive Bayes, bagging, random forests, logistic regression, and the like.

Accordingly, as depicted in FIG. 4, an ensemble 400 of such AI/ML algorithms can be utilized. As provided herein, the ensemble 400 AI/ML combinations and/or iterations can enable a more robust and accurate analysis of the genomic data, among other types of computational analysis discussed herein. In some embodiments, the ensemble model can include, but is not limited to, an area under curve formula, a R matrix (e.g., a matrix of applied algorithms, as depicted in the example of FIG. 4), and p-value formula (e.g., a statistical and/or predictive measurement value formula). Thus, in some embodiments, the ensemble enables an increased accuracy (or greater accuracy) than a NIR (no information rate), as one of skill in the art would understand from the disclosure herein.

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
c. apply the trained model to process the newly-received input data,
f. optionally, and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

In some embodiments, the analysis and determinations of Step 306-308 can include any non-linear ensemble 400 of AI/ML models, as depicted in FIG. 4. For example, such non-linear combination can include, but is not limited to, an ensemble tree with bagging model, eXtreme Gradient Boosting, a random forest model, a SVM with a Gaussian kernel model, an elastic net logistic regression, at least power in alpha band and power in beta band, and the like, and/or some combination thereof. In some embodiments, features of the analyzed data can include, but are not limited to, at least BSR, standard deviation of FM, SVDE and FD.

According to some embodiments, based on a type of data (from Step 302) that is retrieved (in Step 304), specific algorithms may be selected, which as discussed above, can be tuned and/or configured for specific types, formats and/or quantities of data and computational analysis based therefrom. In some embodiments, an AI/ML model may be applied to determine a type, identity and/or quantity of AI/ML algorithms to leverage as part of the ensemble 400. For example, for a particular BSR, deviation and SVDE, algorithms X and Y may be selected to iteratively operate in a non-linear manner on the subject data. Thus, different AI/ML algorithms may specialized in recognizing part of a solution; therefore, a combinational approach can enable the identification of a full solution.

According to some embodiments, by way of a non-limiting example, a custom ensemble of algorithms (an example of which is visually depicted in FIG. 4) can be used with various types of data and model training targets. For example, a customized AI/ML based ensemble 400 can be applied to, but not limited to: i) clinical patient mutation data, which can include information corresponding to cluster-derived modules and pathway scores, and data corresponding to a categorical training target of patient response based on RECIST (Response evaluation criteria in solid tumors); ii) clinical patient mutation data from The Cancer Genome Atlas (TCGA) Glioblastoma cohort, which can include information corresponding to cluster-derived modules and pathway scores, and data corresponding to a training target of a patient response based on increased survival being above or below median survival; iii) pre-clinical cellular mutation data with cells from the National Cancer Institute (NCI) NCI60 panel, without inclusion of cluster-derived modules and pathway scores, and with a regression training target of the IC50 (e.g., in −log IO IC50 (M) format) of cells treated with a drug; and iv) mutation data prepared from tumor samples, which can be scored in a binary fashion (e.g., if a mutation occurs, the sample is given a 1, otherwise 0). In some embodiments, the mutation data having synonymous mutations may not be duplicated, and therefore can be executed (e.g., synonymous mutations for a patient, genome and/or drug, for example, may be excluded and/or filtered out from the analyzed data).

Accordingly, in some embodiments, Step 308, therefore, can involve the determination of a score (or metric) for the data, thereby providing an indication as to a value and/or qualification of the binary mutation. As discussed above, such scoring can be based on and/or involve the feature engineering processing discussed above. As provided below, this scoring can be utilized to group or cluster the data for subsequent drug responsiveness analysis.

In Step 310, engine 200 can analyze the determined data from Step 308 (e.g., the output from the ensemble 400 analysis—for example, the binary mutations determined), and in Step 312, determine a clustering of the data. According to some embodiments, such clustering can target (or group) one or more genes to identify features (e.g., pathway features, for example) that are correlated with the responsiveness to a drug or drugs and to obtain hierarchical clustered data, as discussed infra.

According to some embodiments, such analysis and clustering determination can be based on and/or involve the ensemble determination and application discussed above respective to Steps 306-308. For example, based on a type of binary mutations, feature engineering and/or criteria for clustering (e.g., a quantity and/or quality of clustered data sets), particular AI/ML algorithms may be identified and executed in a non-linear and/or sequential manner.

By way of a non-limiting example, engine 200 can cluster the data by grouping (or “cutting” or filtering data according to a specific criteria (e.g., a height or desired number of clusters, for example). For example, if the criterial corresponds to k clusters, where k=40, then out of 400 binary mutations analyzed, 40 clusters can be generated.

According to some embodiments, engine 200 may perform the clustering in Step 312 based on a set of features and/or biological pathways. In some embodiments, data can be clustered using hierarchical clustering to make a cluster or group, which enables the reduction of the sparsity of data. In some embodiments, the data can be clustered based on their determined scores from Step 308, as discussed supra. For example, mutations within a range of scoring can be clustered together. In another non-limiting example, mutations can be grouped when their corresponding scores equal a sum associated with and/or indicating a particular biological pathway and/or mutations in such biological pathways.

According to some embodiments, in yet another non-limiting example, with 100-1100 columns of different features initially, the initial data can be culled or reduced using iterative feature reduction via an algorithm that is trained with hyperparameters tuned to optimize performance in accordance with a set of constraints. In some embodiments, upon evaluation of this analysis, feature reduction can be performed when the evaluation fails to satisfy performance threshold. In some embodiments, such feature reduction can occurring in a stepwise manner until a the total features (e.g., clusters) are less than or equal to a feature threshold (e.g., for example, feature reduction continues with stepwise reduction by 90% or 50% of features until a number of usable features are a predetermined percentage (e.g., 10%) of the total number of features (e.g., prior to reduction)). Accordingly, such feature reduction can prevent overfitting, thereby enabling the ensemble mechanisms and methodologies discussed herein to be efficiently applied while ensuring an improved accuracy is evidenced upon implementation.

Upon clustering of the data in Step 312, engine 200 can identify a target drug and target sample, as in Step 314, whereby in Step 316, the clustered/reduced feature set (from Step 312) can be fed to an ensemble algorithm model. In some embodiments, the target sample corresponds to and/or is represented by the clustered data. In some embodiments, the target sample and/or drug can be identified from the database, as discussed above.

According to some embodiments, the ensemble algorithm model can be configured and/or determined (e.g., which algorithms are part of the ensemble) based on a type of clustering and/or quantity of clusters, which can be determined in a similar manner as discussed above. In some embodiments, the compiled ensemble from prior steps in Process 300 can be identified and selected for usage.

Thus, in Step 316, engine 200 can identify and/or determine the ensemble, whereby upon its execution, a determination regarding the target drug's responsiveness can be performed. Thus, in Step 316, engine 200 can determine how a drug will operate (e.g., how effective it is) against a condition (e.g., represented via a target sample). Such data can then be stored in a database, as in Step 318. In some embodiments, such responsiveness data can be recursively fed back to the ensemble (or at least a portion of algorithms used in processing of Process 300) to effectuate further training of the models implemented therein.

According to some embodiments, an input a list of algorithms “R” as, for example, “algo_name1”, “algo_name2”, with possible repeats (e.g., an ensemble can tune the same algorithm in different ways and perform differently, and can both possibly contribute to an ensemble), can be utilized as information when generating the ensembles discussed above and/or can be associated with the stored information so as to provide indicators as to the analysis that resulted in the responsiveness determinations. In some embodiments, with regarding to training and/or retraining (e.g., via Step 318), some customized steps can be done to make different data splits in the training data and 25-50% of the training data (optional) so the ensemble does not overfit (as discussed above). In some embodiments, different algorithms can be trained and saved in a “list” object in R, then predictions using the models can be saved, which can be added to the data with patient samples and mutations and clusters. Thus, upon storage and training of the ensemble, a “final” algorithm that was selected for Step 316 can be used to do AI/ML training on a new training set which includes the predictions stored in Step 318.

FIG. 7 is a schematic diagram illustrating a client device showing an example embodiment of a client device that may be used within the present disclosure. Client device 700 may include many more or fewer components than those shown in FIG. 7. However, the components shown are sufficient to disclose an illustrative embodiment for implementing the present disclosure. Client device 700 may represent, for example, UE 102 discussed above at least in relation to FIG. 1.

As shown in the figure, in some embodiments, Client device 700 includes a processing unit (CPU) 722 in communication with a mass memory 730 via a bus 724. Client device 700 also includes a power supply 726, one or more network interfaces 750, an audio interface 752, a display 754, a keypad 756, an illuminator 758, an input/output interface 760, a haptic interface 762, an optional global positioning systems (GPS) receiver 764 and a camera(s) or other optical, thermal or electromagnetic sensors 766. Device 700 can include one camera/sensor 766, or a plurality of cameras/sensors 766, as understood by those of skill in the art. Power supply 726 provides power to Client device 700.

Client device 700 may optionally communicate with a base station (not shown), or directly with another computing device. In some embodiments, network interface 750 is sometimes known as a transceiver, transceiving device, or network interface card (NIC).

Audio interface 752 is arranged to produce and receive audio signals such as the sound of a human voice in some embodiments. Display 754 may be a liquid crystal display (LCD), gas plasma, light emitting diode (LED), or any other type of display used with a computing device. Display 754 may also include a touch sensitive screen arranged to receive input from an object such as a stylus or a digit from a human hand.

Keypad 756 may include any input device arranged to receive input from a user. Illuminator 758 may provide a status indication and/or provide light.

Client device 700 also includes input/output interface 760 for communicating with external. Input/output interface 760 can utilize one or more communication technologies, such as USB, infrared, Bluetooth™, or the like in some embodiments. Haptic interface 762 is arranged to provide tactile feedback to a user of the client device.

Optional GPS transceiver 764 can determine the physical coordinates of Client device 700 on the surface of the Earth, which typically outputs a location as latitude and longitude values. GPS transceiver 764 can also employ other geo-positioning mechanisms, including, but not limited to, triangulation, assisted GPS (AGPS), E-OTD, CI, SAI, ETA, BSS or the like, to further determine the physical location of client device 700 on the surface of the Earth. In one embodiment, however, Client device may through other components, provide other information that may be employed to determine a physical location of the device, including for example, a MAC address, Internet Protocol (IP) address, or the like.

Mass memory 730 includes a RAM 732, a ROM 734, and other storage means. Mass memory 730 illustrates another example of computer storage media for storage of information such as computer readable instructions, data structures, program modules or other data. Mass memory 730 stores a basic input/output system (“BIOS”) 740 for controlling low-level operation of Client device 700. The mass memory also stores an operating system 741 for controlling the operation of Client device 700.

Memory 730 further includes one or more data stores, which can be utilized by Client device 700 to store, among other things, applications 742 and/or other information or data. For example, data stores may be employed to store information that describes various capabilities of Client device 700. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 700.

Applications 742 may include computer executable instructions which, when executed by Client device 700, transmit, receive, and/or otherwise process audio, video, images, and enable telecommunication with a server and/or another user of another client device. Applications 742 may further include a client that is configured to send, to receive, and/or to otherwise process gaming, goods/services and/or other forms of data, messages and content hosted and provided by the platform associated with engine 200 and its affiliates.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user” or “patient” should be understood to refer to a user of an application or applications as described herein and/or a consumer of data supplied by a data provider. By way of example, and not limitation, the term “user” or “patient” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising:
identifying, by a device, genomic mutation data;
analyzing, by the device, the genomic mutation data using an ensemble model trained on prior mutation data;
determining, by the device, based on the ensemble analysis, a set of binary mutations relevant to drug responsiveness;
clustering, by the device, the set of binary mutations based on similarity metrics derived from mutation data;
analyzing, by the device, the clustered binary mutations based on information related to an identified drug;
determining, by the device, a responsiveness of each cluster to the identified drug based on analysis of the clustered binary mutations; and
storing, by the device, the responsiveness score and associated genomic data in a structured database for further analysis and retrieval.

2. The method of claim 1, further comprising:
determining a set of algorithms based on the analysis of the genomic mutation data; and
compiling, based on the determined set of algorithms, the ensemble model.

3. The method of claim 2, wherein he ensemble model comprises non-linear machine learning algorithms optimized for mutation clustering and drug responsiveness prediction.

4. The method of claim 2, wherein the information stored in the database includes the identified binary mutations, the drug responsiveness score, and the algorithms used in the ensemble model.

5. The method of claim 1, wherein the ensemble model is continuously updated based on feedback from the determined drug responsiveness data.

6. The method of claim 1, wherein the ensemble model uses an optimization framework incorporating metrics such as area under curve (AUC), R-matrix, and statistical significance (p-value) for accuracy in drug-response predictions.

7. The method of claim 1, wherein the determined drug responsiveness score is a quantitative metric indicating how a drug modulates a specific binary mutation within a cluster.

8. The method of claim 1, further comprising:
identifying a type of each of the binary mutations;
performing the clustering of the binary mutations based on the identified type, wherein each cluster corresponds to a particular type of binary mutation.

9. The method of claim 1, further comprising:
iteratively, as a stepwise function, executing feature reduction on the set of binary mutation data until a set of features for the clustering satisfies a threshold, wherein the clustering is based on the feature reduction.

10. The method of claim 9, wherein the feature reduction process is governed by machine learning constraints to ensure consistency in clustering and responsiveness prediction.

11. The method of claim 1, further comprising:
identifying a type of data and a corresponding database;
querying the database; and
retrieving the genomic mutation data based on the query, wherein the identification of the genomic mutation data is based on the retrieval.

12. A device comprising:
a processor configured to:
retrieve genomic mutation data from a database;
analyze the genomic mutation data using an ensemble model;
determine, based on the analysis, a set of binary mutations;
cluster the set of binary mutations;
analyze the clusters with respect to drug responsiveness;
determine a responsiveness of each cluster to the identified drug based on analysis of the clustered binary mutations; and
store information within a database indicating the determined responsiveness.

13. The device of claim 12, wherein the processor is further configured to:
determine a set of algorithms based on the analysis of the genomic mutation data; and
compile, based on the determined set of algorithms, the ensemble model, wherein the ensemble model comprises a non-linear execution of the determined set of algorithms.

14. The device of claim 12, wherein the processor is further configured to:
identify a type of each of the binary mutations;
perform the clustering of the binary mutations based on the identified type, wherein each cluster corresponds to a particular type of binary mutation.

15. The device of claim 12, wherein the processor is further configured to:
iteratively, as a stepwise function, execute feature reduction on the set of binary mutation data until a set of features for the clustering satisfies a threshold, wherein the clustering is based on the feature reduction, wherein the feature reduction is based on a set of constraints.

16. The device of claim 12, wherein the processor is further configured to:
identify a type of data and a corresponding database;
query the database; and
retrieve the genomic mutation data based on the query, wherein the identification of the genomic mutation data is based on the retrieval.

17. A non-transitory computer-readable storage medium tangibly encoded with computer-executable instructions that when executed by a device, performs a method comprising:
identifying, by the device, genomic mutation data;
analyzing, by the device, via an ensemble model, the genomic mutation data;
determining, by the device, based on the analysis, a set of binary mutations;
clustering, by the device, the set of binary mutations;
analyzing, by the device, the clustered binary mutations based on information related to an identified drug;
determining, by the device, a responsiveness of each cluster to the identified drug based on analysis of the clustered binary mutations; and
storing, by the device, information within a database indicating the determined responsiveness.

18. The non-transitory computer-readable storage medium of claim 17, further comprising:
determining a set of algorithms based on the analysis of the genomic mutation data; and
compiling, based on the determined set of algorithms, the ensemble model, wherein the ensemble model comprises a non-linear execution of the determined set of algorithms.

19. The non-transitory computer-readable storage medium of claim 17, further comprising:
identifying a type of each of the binary mutations;
performing the clustering of the binary mutations based on the identified type, wherein each cluster corresponds to a particular type of binary mutation.

20. The non-transitory computer-readable storage medium of claim 17, further comprising:
iteratively, as a stepwise function, executing feature reduction on the set of binary mutation data until a set of features for the clustering satisfies a threshold, wherein the clustering is based on the feature reduction, wherein the feature reduction is based on a set of constraints.
